(19) United States
(12) Patent Application Publication
(10) Pub. No.: US 2003/0119174 A1
(43) Pub. Date: Jun. 26, 2003

(54) Pencillin biosensor

(76) Inventors: In Suk Han, Sandy, UT (US); Man-Hee Han, Salt Lake City, UT (US); Seok Lew, Salt Lake City, UT (US)

Correspondence Address: Robert Mallinckrodt, M-Biotech, Inc., Suite C, 2411 South 1070 West, Salt Lake City, UT, 84119, US

(21) Appl. No.: 10/028,079

(22) Filed: Dec. 21, 2001

(51) Int. Cl. C12Q 1/34

(52) U.S. Cl. 435/287.1; 435/287.5; 435/287.7; 435/18

(57) ABSTRACT

The penicillin biosensor (10) has a pH-sensitive polymeric hydrogel (30) in a rigid enclosure (20). The hydrogel includes an immobilized enzyme such as penicillinase. The enzyme catalyzes a chemical reaction consuming penicillin and producing penicillic acid. The hydrogel changes its osmotic pressure in proportion to the concentration of the penicillic acid. By measuring the change in osmotic pressure with a pressure transducer (40), the biosensor (10) is able to accurately measure the concentration of penicillin. A battery (64) powered monitoring device, connected to the biosensor (10) through electrical wires, is operably programmed to display the penicillin concentration in a computer (62) as well as to activate LED or buzzer as an alert in case that the measured concentration of penicillin is over the threshold concentration.

### BACKGROUND OF THE INVENTION

1. FIELD OF THE INVENTION

This invention relates to sensors for detecting penicillin in a fluid.

2. DESCRIPTION OF RELATED ART

Applicants make reference to U.S. Pat. No. 6,268,161 issued Jul. 31, 2001, entitled Biosensor, which is directed to a sensor for measuring concentration of organic molecules. This application has a common inventor to the patent and this application and the patent are assigned to a common assignee.

A major problem in the U.S. dairy industry is ensuring that the milk supply does not contain significant levels of penicillin residues, which cause severe allergic reactions including anaphylactic shock in about 10 to 20% of the population. The presence of penicillin residues in milk is due to the widespread use of penicillin, ampicillin, cephapirin and amoxicillin for treatment of mastitis.

Because of this, the National Conference on Interstate Milk Shipment passed a resolution which modified the Pasteurized Milk Ordinance in 1991 to require screening of all bulk milk for penicillin residues before entering the food supply. As a result, several million penicillin assays are performed every year on cattle milk in US dairy farms.

The penicillin family, which includes cephalosporins, carbapenems, and monobactams, is characterized by the presence of a beta-lactam ring, which is responsible for the antimicrobial activity, inhibiting bacterial cell wall mucopeptide synthesis. To date, 17 screening tests for penicillin residues are available, but only two are in wide use in the dairy industry. One of these is the Bacillus stearothermophilus disc assay (BSDA), which is essentially a microbiological diffusion assay based on inhibition of bacterial growth. The second widely used test involves ELISA immunoassay for the penicillin residues.

However, these methods for penicillin testing of milk are relatively expensive, due to disposable strips and disposable test kits. Additionally, they produce biohazard wastes which must be autoclaved. It also makes it difficult to spot-check individual cows.

There is thus a tremendous need for a better, more economical penicillin testing method, and in particular for a portable, sensitive, reliable biosensor which could be used on-site (in the barns) by non-laboratory-trained personnel.

### SUMMARY OF THE INVENTION

The present invention provides a biosensor for measuring the concentration of penicillin in a fluid. The biosensor includes a pH-sensitive polymeric hydrogel in a rigid enclosure. The hydrogel includes an immobilized enzyme such as penicillinase. The enzyme catalyzes a chemical reaction consuming the penicillin and producing penicillic acid. The hydrogel changes its osmotic pressure in proportion to the concentration of penicillic acid. By measuring the change in osmotic pressure with a means for measuring, preferably a pressure transducer 40, the biosensor is able to accurately measure the concentration of the penicillin. A signal monitor device 62 is operably engaged with the means for measuring. The device displays penicillin concentration by converting an input signal from the means for measuring to penicillin concentration. A primary objective of the present invention is to provide a biosensor that is quick and easy to use for an unskillful user to assay penicillin concentration. Another objective is to provide a biosensor that is extremely sensitive to the very low concentration of penicillin. A further objective is to provide a biosensor that relies on change in pH to measure the penicillin. Other features and advantages of the present invention will become apparent from the following more detailed description, taken in conjunction with the accompanying drawings, which illustrate, by way of example, the principles of the invention.

The present invention relates to a novel biosensor for penicillin analysis in a fluid such as whole blood or milk. This biosensor is fast, reliable, portable, inexpensive, and does not generate hazardous wastes needing specialized disposal.

The penicillin biosensor provides real-time continuous monitoring of penicillin levels in a fluid, which is not possible with the present assay methods. Because the biosensor is extremely quick and easy to use (the user need merely insert the probe in a fluid sample and read the penicillin level off the display), it will be especially valuable for spot-checking of individual cows or small batches of milk, before commingling. The biosensor is relatively simple, with few moving parts, which reduces the cost and increases reliability and robustness. The cost for assaying the penicillin using the biosensor of the present invention is inexpensive because the replacement cost only is spent when replacing hydrogel in the probe, not the associated electronics. Large numbers of sample can be monitored with single probe over several days without changing the hydrogel, depending only on the active life of the enzyme. Thus, the biosensor with replaceable probe is reliable, rugged, durable, and easy-to-operate with low cost.

Furthermore, the principles on which this biosensor is based are not restricted to penicillin detection. This novel class of biosensor has the potential to be adapted for many other kinds of food-testing applications.

### BRIEF DESCRIPTION OF THE DRAWING

FIG. 1 is a vertical sectional view of the preferred embodiment of the present invention, showing a biosensor that is electronically attached to a signal monitor;

FIG. 2 is a vertical sectional view of the preferred embodiment of the present invention, showing penicillin diffusing into the hydrogel, causing the hydrogel to swell and causing the pressure transducer to signal to a signal monitor through electrical wires;

FIG. 3 is a vertical sectional view of the transducer;

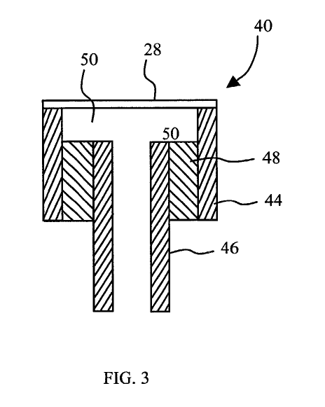

FIG. 4 is a vertical sectional view of the transducer including the preferred circuit board having a diode quad bridge circuit; and

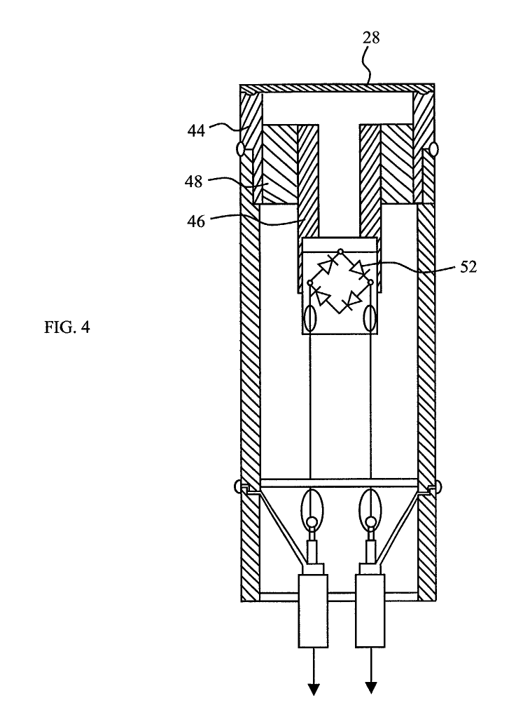

FIG. 5 is a system diagram of a signal monitor and a biosensor.

### DETAILED DESCRIPTION OF THE INVENTION

The above described drawing figures illustrate the invention, a biosensor 10 for measuring the concentration of penicillin in a fluid. In its broadest description, the biosensor 10 uses a special polymeric hydrogel that changes its osmotic pressure in proportion to the concentration of a penicillic acid; an enzyme immobilized in the hydrogel 30, the enzyme catalyzing a chemical reaction consuming penicillin and producing the penicillic acid, thereby causing the hydrogel to change its osmotic pressure; a means for measuring 40 the osmotic pressure of the hydrogel 30; and a signal monitor 62 for monitoring the concentration of the penicillin based on the measured osmotic pressure of the hydrogel 30. In its preferred embodiment, the biosensor includes a rigid, biocompatible enclosure 20 having semipermeable membrane 26 covering an open end 22 a flexible diaphragm 28 between the semipermeable membrane 26 and the closed end 24, and a polymeric hydrogel enclosed therebetween, the hydrogel including moieties that cause the hydrogel 30 to change its osmotic pressure in proportion to the pH of the hydrogel 30. The biosensor 10 is designed for measuring penicillin levels. In this embodiment, the biosensor 10 uses penicillinase as the enzyme immobilized in the hydrogel 30. The means for measuring the osmotic pressure of the hydrogel is preferably a pressure transducer 40 operably associated with the flexible diaphragm 28. The change in the osmotic pressure of the hydrogel that is proportion to the penicillin concentration in a fluid is converted to electrical voltage through the pressure transducer. While the pressure transducer 40 is currently the preferred tool for measuring changes in the osmotic pressure of the hydrogel 30, those skilled in the art can devise alternative means of measuring changes in the osmotic pressure of the hydrogel 30. An alternative method is to use a piezoresistive sensor in place of the pressure transducer 40.

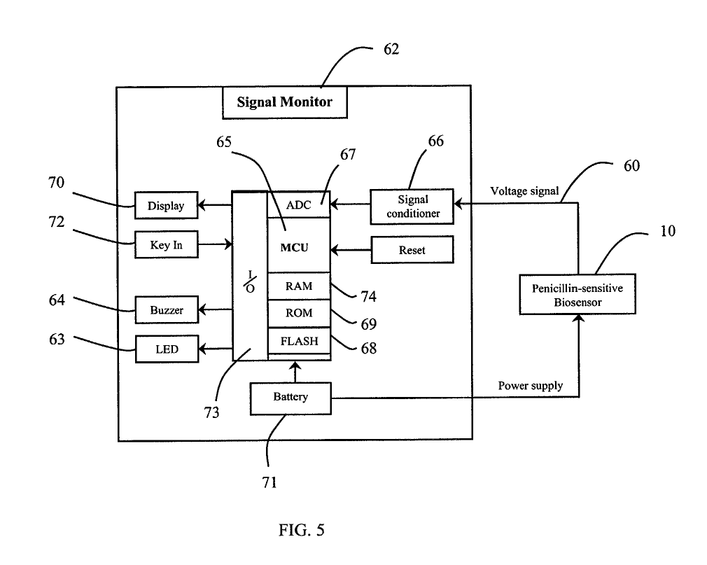

The pressure transducer 40 is directly connected through electric wires 60 to a signal monitor 62 which works for monitoring the penicillin concentrations by using the signal from the penicillin-sensitive biosensor 10. The signal monitor 62 is preferably a digital circuit, so that a certain pre-determined reference value can be programmed into the monitor 62 and the measured value can be compared with the reference value. If the measured value is over the threshold, the monitor activates LED 63 and/or buzzer 64 as a warning (FIG. 5).

The Enclosure, Semipermeable Membrane, and Diaphragm

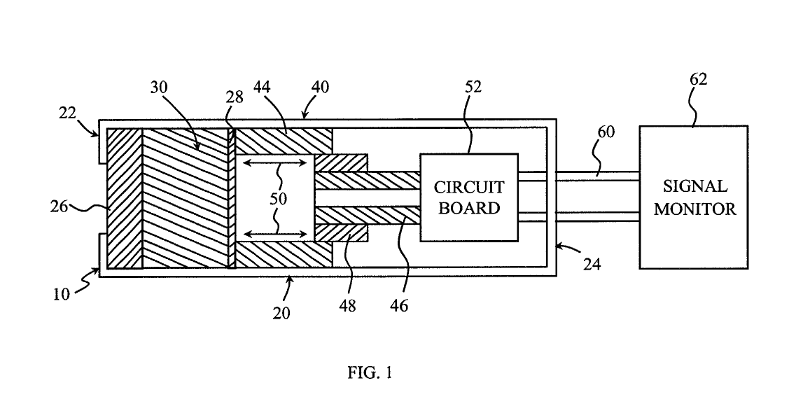

As best shown in FIG. 1, the structure of the biosensor 10 is provided by an enclosure 20, preferably a cylindrical enclosure 20 having an open end and a closed end. The open end 22 is sealed with a semipermeable membrane 26. A flexible diaphragm 28 is mounted between the semipermeable membrane 26 and the closed end 24. The hydrogel 30, described below, is enclosed between the semipermeable membrane 26 and the diaphragm 28. The enclosure 20 is preferably constructed of a rigid, impermeable, and biocompatible material such as stainless steel. The enclosure 20 is preferably cylindrical in shape, the cylinder being approximately 12 mm long and having a diameter of approximately 3 mm.

The semipermeable membrane 26 is permeable to the passage of penicillin and penicillic acid; however, it is impermeable to the passage of cells, proteins, and the hydrogel 30. The semipermeable membrane 26 is preferably made of a material rigid enough to sustain the pressure of a swollen penicillin-sensitive hydrogel 30. A suitable semipermeable material can be selected from, but is not limited to, the following groups of polymers: cellulose acetate, methyl cellulose, polyvinyl alcohol, and polyurethane.

The diaphragm 28 is preferably a flexible but conductive material useful for use with a transducer 40. Such diaphragms are known in the art. The preferred diaphragm 28 is made of an alloy sold under the trademarks KOVAR.TM. or INVAR 36.TM. by Hamilton Technology, Inc., of Lancaster, Pa. The diaphragm 28 is preferably approximately 12.5 um to achieve optimum spot welding and sensitivity. Such a diaphragm is described in Baek S G. Ph.D. Thesis, University of Utah, (1992). The diaphragm 28 is preferably seal welded to the enclosure 20 between the semipermeable membrane 26 and the closed end 24 of the enclosure 20. The hydrogel fills the chamber within the enclosure 20 between the semipermeable membrane 26 and the diaphragm 28. The means for measuring 40 is located in the chamber within the enclosure 20 between the diaphragm 28 and the closed end 24 of the enclosure 20.

pH-Sensitive Hydrogels

Hydrogels are defined as polymeric materials which swell in water and other solvents, absorbing the fluid within the polymer network without dissolving. Hydrophilic hydrogels have a large amount of water content at equilibrium and good biocompatibility. pH-sensitive hydrogels have been the most widely studied of the hydrophilic hydrogels. The pH-sensitive hydrogels are cross-linked to form a stabilized gel with several types of crosslinking forces such as covalent bonds, hydrogen bonds, or hydrophobic interactions. Acidic hydrogels by definition will be ionized and hence swollen at high pH, and uncharged and un-swollen at low pH. Swelling behavior of a basic hydrogel has the opposite dependence on pH. The pH sensitivity is caused by pendant acidic and basic groups such as carboxylic acid, sulfonic acid, primary amine, and quaternary ammonium salts. Carboxylic acid groups for example are charged at high pH and uncharged at low pH, whereas the reverse is true for primary amine groups and quaternary ammonium salts. The transition pH for a given pendant group is determined by the pKa value for that pendant group. Hence by choosing pendant groups with the appropriate pKa values, a hydrophilic hydrogel can be constructed which can be ionized reversibly in response to any level of pH stimuli leading to changes in properties of a gel. In the instant invention, the preferred range of pKa lies between 11 and 3.

The most important property of pH-sensitive hydrogel is its degree of swelling in response to pH. The preferred pH-sensitive hydrogels are derived from a number of polymeric compounds such as: poly(aklyl acrylate), poly(acrylmethacrylate), poly(2-hydroxyethyl methacrylate) (HEMA), poly(2-hydroxypropylmethacrylate) (HPMA), poly(acrylamide), poly(N-vinyl pyrrolidone), poly(vinyl alcohol) (PVA), polyethylene oxide (PEO), poly(etherurethane), and polyelectrolyte. The monomers used to synthesize the homopolymers just listed can also be used in various combinations to form copolymers. pH-sensitive hydrogels formed from these polymers reversibly contract and dilate upon addition of acid and alkaline alternately. It has been shown that the response to a pH change can be very fast and reversible after abrupt changes in pH for poly(methyl methacrylate-co-N,N-dimethylaminoethyl methacrylate) hydrogels. Specific combinations of these compounds can be devised by those skilled in the art to meet the requirements of a specific type of biosensor.

Factors Influencing the Degree of Swelling of pH-Sensitive Hydrogels

The equilibrium degrees of swelling and the conformation changes of pH-sensitive hydrogels are influenced by several factors such as the charge of the ionic monomer, pKa of the ionizable group, concentrations of ionizable pendant group in the network, pH, ionic strength, the dielectric constant of the medium, crosslinking density, hydrophilicity and hydrophobicity of polymer backbone. These factors are discussed in Helle Bronsdted and Jindrich Kopecek, pH-Sensitive Hydrogel; Characteristics And Potential In Drug Delivery in Properties, Preparation, and Application (Edited by Ronald S. Harland and Robert K. Prudhornme), 1992.

The charge of the ionic monomer influences the conformational changes of pH-sensitive hydrogels. An acidic hydrogel will be uncharged at low pH, but it will be ionized at the high pH. Thus, the equilibrium degree of swelling will increase when pH is enhanced in a hydrogel containing acidic pendant groups. Swelling of a basic hydrogel has the opposite dependence on pH. The hydrogels which are based on methacrylic acid, sulfoxyethyl methacrylate, HEMA, and HPMA, and have been generally used to obtain acid, basic, and ampholytic gels. Swelling as a function of the type of ionic groups has been studied. The pKa value of pendant ionizable groups in the gel is shown to influence the pH-swelling curve. A decrease in the pKa value of a basic ionizable group shifts the curve toward lower pH. It has been demonstrated that the swelling response is most sensitive to pH at a pH value close to the pKa value of the ionizable group of the hydrogel. The concentration of ionizable monomers in the hydrogel is significant to the swelling and pH-sensitivity of the gel. This effect depends on the relative hydrophilicity of the ionizable monomer compared to the neutral co-monomer. The hydrophobicity and hydrophilicity of the backbone of the pH-sensitive polymer affects swelling. It has been shown that increasing hydrophobicity of the polymer backbone decreases the pH-sensitivity of the copolymer poly(n-alkyl methacrylate-co-N,N-dimethylaminoethyl methacrylate) and copolymer styrene and 4-vinyl pyridine (VP). Buffer composition and ionic strength affect the swelling of the pH-sensitive hydrogels. Counterions shield charges on the polymeric backbones. The concentration of ions inside and outside of the gel will be equal as well as osmotic pressure inside the gel will decrease when the concentration of ions outside the gel increases. A buffer containing multivalent ion is able to neutralize several charges inside the gel. Cross-linking density is important for pH-sensitive swelling. The equilibrium degree of swelling will be restricted by an increased cross-linking density. This effect is more pronounced if the gel is ionized by a pH change. The network properties of the hydrogels are mainly influenced by the synthesis variables, particularly chemical composition and cross-linking density. Thus, chemical composition and synthesis conditions are important when attempting to control the equilibrium swelling properties of the gels.

The preferred penicillin biosensor will use a pH-sensitive hydrogel which includes copolymers synthesized from various types of methacrylate derived monomers by free radical solution polymerization. These copolymers are tough, flexible polymers rather than soft gels. For example, the swelling of gels which are copolymers of N,N-diethyl-aminoethyl methacrylate (DEAMA) and 2-hydroxypropylmethylacrylate (HPMA) increases with decreasing pH of the medium. This has been shown in Ishihara K. Kobayashi M. Ishimaru N. Shinohara I. Poly J. 16:625-631, (1984), hereby incorporated by reference. By contrast, the water content of the HEMA homopolymer was independent of the pH of the medium. Thus the change in water content with pH of the HPMA copolymer hydrogel resulted from the introduction of the DEAMA moiety. The DEAMA moiety is considered to be protonated when the pH of the medium decreases, which increases the hydrophilicity of the DEAMA moiety and the hydrogel. The water content of DEAMA and HPMA copolymer hydrogel is reversible with respect to pH changes.

Penicillinase Enzyme

The polymeric hydrogel 30 of this invention includes a supply of immobilized penicillinase enzyme. The penicillinase enzyme catalyzes a chemical reaction in the presence of penicillin. The chemical reaction consumes the penicillin and produces penicillic acid. As described below, the penicillic acid causes the hydrogel to change its swelling pressure and swelling pressure in proportion to the concentration of the penicillin.

pH-Sensitive Hydrogels Containing Penicillinase Enzyme

As discussed above, the general combination of a polymeric hydrogel 30 and penicillinase enzyme is what is important to this aspect of the invention. In the preferred biosensor 10, testing for penicillin, immobilization of penicillinase by matrix entrapment in the gel is simpler and more reproducible than other techniques, such as surface immobilization technique, and hence is the preferred method of immobilizing penicillinase in the penicillin biosensor 10. Besides, the penicillinase can be chemically immobilized by conjugating of the penicillinase enzyme into polymer backbones of the hydrogel system.

Penicillin reacts with penicillinase to produce penicillic acid, according to the following formula:

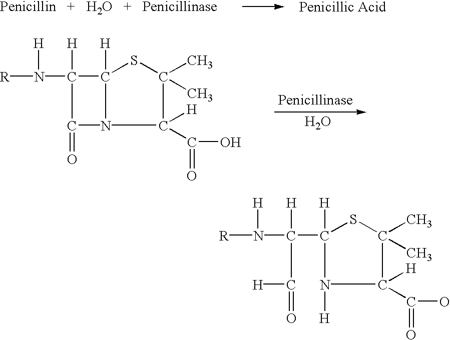

As show in above, penicillinase hydrolyzes the beta-lactam ring structure of the cyclic amide bond, producing a carboxylic acid group and decreasing the ambient pH.

To function in the invention, the hydrogel is preferably pH-sensitive co-polymeric gel that contains immobilized penicillinase to act as a sensor of penicillin, because according to the reaction given earlier, the penicillin is converted to penicillic acid which lowers the pH. The penicillinase for this reaction is very highly specific for penicillin resulting in production of penicillic acid in the presence of penicillin.

Some examples of appropriate systems with penicillinase immobilization are hydrogels 30 based on a collagen-based copolymer, an acrylic-based copolymer, a HPMA-based copolymer, and a HEMA-based copolymer. These hydrogels 30 are sufficiently permeable to penicillin, but not to high molecular weight proteins. The permeability of penicillin in the polymeric hydrogel 30 can be controlled by changing the ratio of monomer compositions such as crosslinkers in the copolymer. The copolymers used to make the pH-sensitive hygrogels 30 contain a certain number of amine groups or carboxylic groups which are involved in the swelling process. These pH-sensitive hydrogels containing penicillinase swell in the presence of penicillin and greatly increase their water content. The penicillinase converts penicillin to penicillic acid. In a basic hydrogel, the penicillic acid protonates the amine groups on the copolymer resulting in production of a charged hydrogel 30 network. The charged amine enhances electrostatic repulsive forces and hydrophilicity in the hydrogel promoting an increase in the hydrogel 30 swelling. The water content of pH-sensitive hydrogels containing pendant tertiary amino groups is drastically increased by the enzymatic conversion of penicillin which produces penicillic acid and lowers the local pH value. The swelling rates of penicillin responsive pH-sensitive hydrogels are dependent on the penicillin concentration in the hydrogel.

Pressure Transducer

The biosensor includes a means for measuring 40 the osmotic pressure of the hydrogel 30. As shown in FIG. 1, the means for measurement is preferably a pressure transducer 40. Pressure transducers are known in the art and those skilled in the field can construct a transducer to the specific needs of the biosensor 10. An example of a transducer is disclosed in Harrison D R, Dimeff J. Rev. Sci. Instrum. 44:1468-1472, (1973) and Harrison et al., U.S. Pat. No. 3,869,676, titled Diode-Quad Bridge Circuit Means, hereby incorporated by reference. In its most preferred embodiment, the means for measuring 40 is a capacitive pressure transducer 40 associated with the flexible diaphragm 28 described above. The preferred transducer 40 includes a first electrode 44 and a second electrode 46, the first and second electrodes 44 and 46 being separated by an insulator 48. In its preferred embodiment, the first and second electrodes 44 and 46, as well as the insulator 48, are coaxially aligned cylinders. The flexible diaphragm 28 is preferably welded to the top of the first conductor 44, converting the diaphragm 28 into one of the electrodes of a capacitor portion of the transducer 40. The first electrode 44 is connected to the diaphragm 28, and the diaphragm 28 is separated from the second electrode 46 by an air gap 50. Since the diaphragm 28 is in mechanical contact with the hydrogel 30, the diaphragm 28 deflects in response to changes in the osmotic pressure of the hydrogel 30, thereby changing the size of the air gap 50 between the second electrode 46 and the diaphragm 28, thereby changing the value of the capacitance. The value of the capacitance change is detected remotely, preferably using a diode quad bridge circuit 52. These pressure transducers 40 have been successfully used to measure pressure changes in flowing polymeric liquids as small as one Pascal.

Examples of alternative transducers are described in Takaki, U.S. Pat. Nos. 5,711,291 and Fowler, 5,752,918, hereby incorporated by reference. A more detailed discussion of transducers can be found in the following references, hereby incorporated by reference: Baek S G. Ph.D. Thesis, University of Utah, (1991); Magda J J, Baek S G, Larson R G, DeVries K L. Polymer 32:1794-1797, (1991); Magda J J, Baek S G, Larson R G, DeVries K L. Macromolecules 24:4460-4468, (1991); Magda J J, Lou J, Baek S G. Polymer 32:2000-2009, (1991); Lee C S, Tripp B, Magda J J. Rheologica Acta 31:306-308, (1992); Lee C S, Magda J J, DeVries K L, Mays J W. Macromolecules 25:4744-4750, (1992); Magda J J, Baek S G. Polymer 35:1187-1194, (1994); Lou J. M. S. Thesis, University of Utah, (1992); Fryer T. Biotelemetry III, Academic Press, New York, pp.279-282, (1976); Updike S J, Shults M C, Rhodes R K, Gilligan B J, Luebow J O, von Heimburg D. ASAIO J. 40:157-163, (1994); and Foulds N C, Frew J E, Green M J. Biosensors A Practical Approach (Cass A E G. eds.) IRL Press Oxford University, pp. 116-121, (1990). While a preferred pressure transducer 40 has been described, those skilled in the art can devise other means for measuring 40.

One alternative embodiment includes a piezoresistive pressure transducer. This alternative is considered equivalent to the described invention. The piezoresistive pressure transducer can measure the applied osmotic pressure by way of resistance change in whetstone bridge circuit inside of the pressure transducer, and eventually provide a voltage signal in proportion to the osmotic pressure. The piezoresistive pressure die is available from pressure transducer manufacturers, and P1300 die from NovaTRW, which is suitable for measuring low pressure, can be used for developing the biosensor 10, when the amount of penicillin is expected to be very small and the hydrogel swelling pressure is low accordingly.

Signal Monitor of Penicillin Concentration Level

In order to monitor the electric signal of penicillin level from the biosensor 10, it is appropriate that a low power consuming microprocessor, for example, a 8 bit microprocessor 65 have internal RAM 74, ROM 69, Flash memory 68, ADC (Analog-digital converter) 67, and I/O 73.

FIG. 5 shows the system diagram for the signal monitor. The system measures the input signal value as programmed in ROM 69. The signal from the penicillin-sensitive biosensor 10 is amplified in signal conditioner 66 and converted into digital value in ADC 67 and compared with the reference value stored in the flash memory 68. As the signal exceeds the pre-determined reference values, the system triggers LED 63 and/or buzzer 64. The current penicillin level is obtained from calibration curve data of voltage signal vs. concentration of penicillin in a fluid and then displayed in LCD 70. The battery 71 inside the signal monitor provides power to the signal monitor 62 and the biosensor 10. The accumulated signal of penicillin level can be stored in the flash memory 68 and retrieved with the engagement of key input 72 by user.

Method for Using a Biosensor to Measure the Concentration of Penicillin in a Fluid

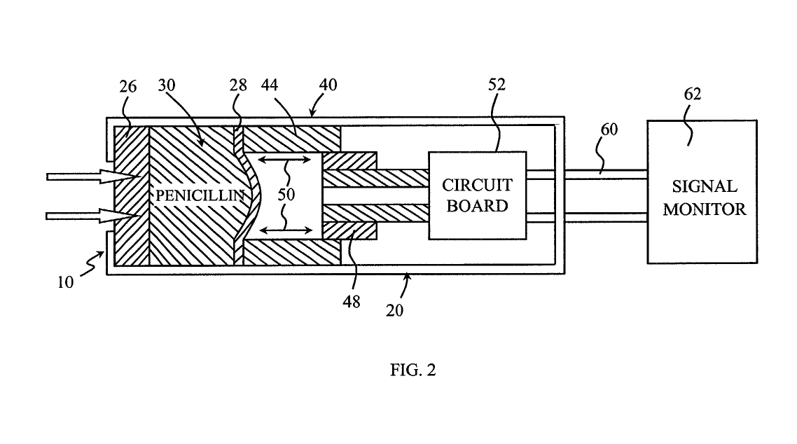

The invention further includes a method for using a biosensor 10 to measure the concentration of penicillin. The method includes the following steps: First, providing a biosensor 10 as described above. An enzyme such as penicillinase is immobilized in the hydrogel 30, preferably using matrix entrapment. The biosensor 10 is preferably first inserted into a control fluid that does not have penicillin. The data generated is then compared to a calibration curve to calibrate the biosensor 10. Once the biosensor 10 is removed and rinsed in the fluid, the biosensor 10 is inserted into the suspected fluid. The penicillin molecules are allowed to diffuse into the polymeric hydrogel 30, causing the penicillinase to catalyze a chemical reaction consuming the penicillin and producing penicillic acid. As described above, the penicillinase enzyme is preferably used to catalyze a reaction in which penicillin are converted into penicillic acid. The production of penicillic acid causes the pH to lower, thereby causing the hydrogel 30 to increase in osmotic pressure and swell, as shown in FIG. 2. This swelling is measured with the means for measuring 40. The means for measuring 40 is preferably a pressure transducer 40. The pressure transducer 40 is used to measure the osmotic pressure of the hydrogel 30, which is proportional to the pH level in the hydrogel 30 (which is proportional to the concentration of the penicillin). Data from the transducer 40 regarding this measurement is then sent to the signal monitor 62.

In addition to the above-described disclosure, it is useful to consider the detailed disclosures made in the following references, hereby incorporated by reference: Atherton H V, Newlander J A. Chemistry and Testing of Dairy Products, AVI publishing company, Inc., fourth edition, 211-217, (1977); Allcock H R, Ambrosio A M. Biomaterials 17:2295-2302, (1996); Batich C D, Yan J, Bucaria Jr C, Elsabee M. Macromolecules 26:4675-4680, (1993); Brannon-Peppas L, Peppas N A. Biomaterials 11:635-644, (1990); Brondsted H, Kopecek J. Polyelectrolyte gels: Properties, Preparation, and Application, Harland R. S. and P. K. Prud homme (eds.), 285-304, (1992); De Moor C P, Doh L, Siegel R A. Biomaterials 12:836-840, (1991); Firestone B A, Siegel R A. J. Biomater Sci. Polym. Ed., 5:433-450, (1994); Foulds N C, Frew J E, Green M J. Biosensors: A Practical Approach (A.E.G. Cass eds.) IRL Press oxford university, 116-121, (1990); Ghandehari H, Kopeckovd P, Yeh P-Y, Kopecek J. Macromol. Chem. Phys. 197:965-980, (1996); Guilbault G G, Suleiman A A, Fatibello-Filho O, Nabirahni M A. in Bioinstrumentation and Biosensors (D.L Wise ed.), Marcel Dekker, 659-692, (1991); Jung D -Y, Magda J J, Han I S. Macromolecules 33:3332-3336, (2000); Khare A R, Peppas N A. Biomaterials 16:559-567, (1995); Siegel R A, Firestone B A. Macromolecule 21:3254-3259, (1988); Siegel R A, Johannes I, Hunt C A, Firestone B A. Pharm. Res. 9:76-81, (1992); Vakkalanka S K, Brazel C S, Peppas N A. J. Biomater. Sci. Polym. Ed. 8:119-129, (1996); Vazquez B, Gurruchaga M, San Roman J. Biomaterials 18:521-526, (1997).

What is claimed is:

1. A biosensor for measuring the concentration of penicillin molecules, the biosensor comprising:
a polymeric hydrogel that changes its osmotic pressure in proportion to the concentration of a penicillic acid;
an penicillinase enzyme immobilized in the hydrogel, the penicillinase enzyme catalyzing a chemical reaction consuming the penicilin molecules and producing the penicllic acid, thereby causing the hydrogel to change its osmotic pressure;
a means for measuring the osmotic pressure of the hydrogel; and a means for reporting the concentration of the penicillin molecule based on the measured osmotic pressure of the hydrogel.

2. The biosensor of claim 1 further comprising an enclosure containing the hydrogel, the enclosure having an open end sealed by a semipermeable membrane that allows water and the penicillin molecules to diffuse into the hydrogel.

3. The biosensor of claim 2 wherein the enclosure further includes a flexible diaphragm, the hydrogel being enclosed between the flexible diaphragm and the semipermeable membrane, the flexible diaphragm working in conjunction with the means for measuring to monitor changes in the osmotic pressure of the hydrogel.

4. The biosensor of claim 1 wherein the hydrogel includes crosslinking that allows the penicillin and water to diffuse into the hydrogel.

5. The biosensor of claim 1 wherein the hydrogel includes pendant groups having a pKa value between 11 and 3.

6. The biosensor of claim 1 wherein the hydrogel is nontoxic, and inert in a fluid.

7. The biosensor of claim 1 wherein the concentration of penicillin molecules is measured in a physiological fluid.

8. The biosensor of claim 7 wherein the physiological fluid is milk.

9. The biosensor of claim 7 wherein the physiological fluid is whole blood.

10. The biosensor of claim 1 wherein the means for measuring is a pressure transducer.

11. The biosensor of claim 1 wherein the pressure transducer is selected from the groups consisting of a capacitive pressure transducer, a piezoelectric transducer, or a piezoresistive transducer.

12. The biosensor of claim 1 wherein the means for measuring is electrically connected to a signal monitor which includes a digital circuit, the digital circuit comparing data from the means for measuring to a calibration curve to calculate the concentration of the penicillin, the signal monitor then reporting the concentration through the digital circuit.

13. The biosensor of claim 12 wherein the digital circuit is programmed such that the signal value transmitted from the means for measuring can be compared with the predetermined reference value.

14. The biosensor of claim 12 wherein the digital circuit uses a low power consuming microprocesser such as a 8 bit microprocessor.

15. The biosensor of claim 14 wherein the microprocessor includes internal RAM, ROM, Flash memory, ADC (analog-digital converter), and I/O.

16. The biosensor of claim 12 wherein the digital circuit includes a signal conditioner for amplifying the input signal transmitted from the means for measuring.

17. The biosensor of claim 12 wherein the digital circuit includes warning equipment which can be activated if the signal value transmitted from the means for measuring exceeds the predetermined reference value.

18. The biosensor of claim 12 wherein the warning equipment is either a LED or a buzzer.

19. The biosensor of claim 12 wherein the digital circuit includes a display device for displaying the signal transmitted from the means for measuring.

20. The biosensor of claim 19 wherein the display device is a LCD.

21. A biosensor for measuring the concentration of penicillin in a fluid, the biosensor comprising:
a rigid, biocompatible enclosure having an open end and a closed end, the open end being covered by a semipermeable membrane; a flexible diaphragm being positioned between the semipermeable membrane and the closed end; and a polymeric hydrogel enclosed between the semipermeable membrane and the diaphragm, the hydrogel including moieties that cause the hydrogel to change its osmotic pressure in proportion to the pH of the hydrogel;
an amount of the enzyme immobilized in the hydrogel;
a pressure transducers selected from the group consisting of capacitance transducers, piezoelectric transducers, and piezoresistive transducers, operatively engaged to the diaphragm.

22. A method for using a biosensor to measure the concentration of penicillin in a fluid, the method comprising the steps of:
a) providing a biosensor comprising:
a rigid enclosure having an open end and a closed end, the open end being covered by a semipermeable membrane; a flexible diaphragm being positioned between the semipermeable membrane and the closed end; and a polymeric hydrogel enclosed between the semipermeable membrane and the diaphragm, the hydrogel including moieties that cause the hydrogel to change its osmotic pressure in proportion to the pH of the hydrogel;
an penicillinase enzyme immobilized in the hydrogel
a means for measuring the osmotic pressure of the hydrogel, the means for measuring being associated with the diaphragm; and
a means for reporting the concentration of the penicillin based on the measured osmotic pressure of the hydrogel;
b) providing a fluid, the fluid containing an amount of the penicillin;
c) inserting the biosensor into the fluid;
d) allowing penicillin to diffuse into the polymeric hydrogel;
e) allowing the penicillinase enzyme to catalyze a chemical reaction consuming the penicillin and producing penicillic acid;
f) measuring the osmotic pressure of the hydrogel with the means for measuring;
g) sending data regarding the osmotic pressure of the hydrogel from the means for measuring to the means for reporting; and
h) reporting the concentration of the penicillin based upon the osmotic pressure of the hydrogel measured by the means for measuring.

23. The method of claim 22 further comprising the steps of:
a′) inserting the biosensor into a fluid;
a″) inserting the biosensor into a control fluid that does not contain penicillin and comparing the data generated to a control curve, thereby calibrating the biosensor; and
